(19) United States
(12) Patent Application Publication
(10) Pub. No.: US 2006/0088501 A1
(43) Pub. Date: Apr. 27, 2006

(54) Duffy antigen receptor for chemokines and use thereof

(75) Inventor: Asok Chaudhuri, Hicksville, NY (US)

Correspondence Address: HOFFMANN & BARON, LLP, 6900 JERICHO TURNPIKE, SYOSSET, NY, 11791, US

(73) Assignee: New York Blood Center, Inc.

(21) Appl. No.: 11/254,222

(22) Filed: Oct. 19, 2005

Related U.S. Application Data
(60) Provisional application No. 60/620,993, filed on Oct. 21, 2004.

(51) Int. Cl. A61K 38/19; G01N 33/567; A61K 39/395

(52) U.S. Cl. 424/85.1; 435/7.2; 424/145.1

(57) ABSTRACT

The invention relates to Duffy antigen receptor for chemokines and uses thereof. In one embodiment, the invention provides a method for screening for drug candidates that inhibit angiogenesis. The method comprises contacting a molecule with a Duffy antigen receptor for chemokines and determining whether the molecule binds to the Duffy antigen receptor for chemokines. In another embodiment, the invention provides a method for inhibiting tumor growth. In yet another embodiment, the invention provides a method for inhibiting angiogenesis. In a further embodiment, the invention provides a method for promoting tumor necrosis.

This application asserts priority to U.S. Provisional Application Ser. No. 60/620,993 filed on Oct. 21, 2004, the specification of which is hereby incorporated by reference in its entirety.

### BACKGROUND OF THE INVENTION

Duffy blood group protein is a member of the seven-transmembrane domain chemokine receptor family (Chaudhuri et al., PNAS, 1993, 90:10793-10797). The protein is capable of binding chemokines, such as CXC and CC chemokines. Therefore, the protein is also referred to as Duffy antigen receptor for chemokines (DARC) (Chaudhuri et al., J Biol Chem. 1994, 269:7835-7838 and Horuk et al., Immunol Today 1994, 15:169-174).

In addition to being expressed on erythroid cells, DARC is also present on endothelial cells. For example, DARC is present on microvascular endothelial cells of several nonerythroid organs such as the liver, lungs, thyroid, and spleen; as well as in epithelial cells of lungs and kidney collecting ducts (Chaudhuri et al., Blood 1997, 89:701-712). Purkinje cells of the brain also contain DARC (Horuk et al., J Leukocyte Biol. 1996, 59:29-38).

Angiogenesis is the process of developing new blood vessels. The process involves the proliferation, migration and tissue infiltration of capillary endothelial cells from pre-existing blood vessels. However, the vascular endothelium is usually quiescent in healthy adults, and its activation is tightly regulated during angiogenesis.

Angiogenesis is important in normal physiological processes including embryonic development, follicular growth, and wound healing, as well as in pathological conditions involving tumor growth, metastasis, abnormal ocular neovascularization, arthritis, psoriasis, and several disorders of the female reproductive system. For example, a growing tumor has been reported to require a rapidly growing vasculature to supply nutrients to the tumor. Thus, regulation and/or inhibition of angiogenesis would be beneficial in pathological conditions, such as cancer.

The ligands of the chemokine receptor family are chemotactic cytokines, also called chemokines. Chemokines are peptides that generally contain four highly conserved Cys residues that form two disulfide bonds (Baggiolini et al., New England Journal of Medicine 1998, 338:436-445). There are several families of chemokines. The two largest families are the CXC chemokines and the CC chemokines. Other chemokine families include the C chemokines and CX₃C chemokines.

The CXC family of chemokines contain two highly conserved Cys residues at the peptide amino terminus separated by any amino acid. Chemokines belonging to the CC family have two Cys residues proximate to one another.

Chemokines induce cell migration and activation by binding to chemokine receptors on a large number of target cells. Chemokines have been reported to be involved in a variety of diseases. For a review, see Luster, New England Journal of Medicine 1998, 338:436.

Several chemokines, including IL-8, have been reported to have a stimulatory effect on angiogenesis (Dawson et al., Blood 2000, 96:1681-1684). For example, Debsaillets et al. (J. Exp. Med. 1997, 186:1201-1212) investigated whether augmented IL-8 would directly and/or indirectly promote angiogenesis by binding to DARC. The authors conclude that further in vivo studies will have to establish whether IL-8 and other angiogenic chemokines are essential contributors to the angiogenic response, and whether this response is mediated by DARC.

Further, U.S. Pat. No. 6,365,356 to Gershengorn report that cell signaling for angiogenesis and angiostasis involves the co-expression of DARC and a chemokine receptor in an endothelial cell. However, the precise function of DARC is not reported.

In addition, a recent article reported an increase in tumor necrosis and a decrease in tumor-associated angiogenesis in tumors expressing DARC. See Addison et al. (BMC Cancer, 2004, 4:28).

Of the above described references that address the role of DARC in angiogenesis, Debsaillets et al. report that further in vivo studies are required; Gershengom does not report the precise function of DARC, and Addison et al. report that tumors expressing DARC exhibit a decrease in tumor-associated angiogenesis. Thus, the role of DARC in angiogenesis is unclear.

There is a need for improved methods for inhibiting angiogensis and tumor growth, and for promoting tumor necrosis. There is also a need for screening methods for discovering drug candidates that inhibit angiogenesis.

### SUMMARY OF THE INVENTION

These and other objectives have been achieved by the present invention which provides a method for screening for drug candidates that inhibit angiogenesis. The method comprises contacting a molecule with a Duffy antigen receptor for chemokines and determining whether the molecule binds to the Duffy antigen receptor for chemokines. Molecules that bind to the Duffy antigen receptor for chemokines are drug candidates that inhibit angiogenesis.

In another embodiment, the invention provides a method for inhibiting tumor growth in a mammal in need thereof. The method comprises administering to the mammal an effective amount of a molecule that inhibits binding of an angiogenic chemokine to Duffy antigen receptor for chemokines.

In yet another embodiment, the invention provides a method for inhibiting angiogenesis in a mammal in need thereof. The method comprises administering to the mammal an effective amount of a molecule that inhibits binding of an angiogenic chemokine to Duffy antigen receptor for chemokines.

In a further embodiment, the invention provides a method for promoting tumor necrosis in a mammal in need thereof. The method comprises administering to the mammal an effective amount of a molecule that inhibits binding of an angiogenic chemokine to Duffy antigen receptor for chemokines.

### BRIEF DESCRIPTION OF THE FIGURES

FIG. 1. Tumor Weights in Dfy (−/−) and Dfy (+/+) Mice.

FIG. 2. LLC injected Tumor Growth In WT [Dfy(+/+)] & DKO Mice. (A) Necrosis in Dfy(−/−) mice are shown with arrow. (B) Tumor in WT mice are more solid and dense compared to that of DKO mice. (C) Histology of tumor tissues showed the density of tumor and more micro capillary formation (arrow) compared to that in DKO mice.

FIG. 3. Effect of Anti-Duffy Antibody in LLC Induced Tumor Growth.

### DETAILED DESCRIPTION OF THE INVENTION

The invention is based on the surprising discovery by the inventor that inhibition of DARC inhibits tumor growth and angiogenesis. The inventor also unexpectedly discovered that inhibitors of DARC promote tumor necrosis.

Inhibiting Tumor Growth

In one embodiment, the invention provides a method for inhibiting tumor growth in a mammal in need thereof. Mammals in need of inhibiting tumor growth are those mammals suffering from a tumor. Any type of tumor that requires angiogenesis can be treated in accordance with the method of the present invention.

A tumor is typically an abnormal mass of tissue or cells which generally results from excessive cell division. The tumor can arise from any tissue or cell. Examples of tissues or cells include epithelial cells, endocrine tissue, bone cells, prostate cells, brain tissue, kidney cells, lung cells, breast tissue, ovarian tissue, colon tissue, retinal tissue, etc. The tumor can be benign (not cancerous) or malignant (cancerous). The method is especially effective when the tumor is malignant.

The method for inhibiting tumor growth comprises administering to the mammal an effective amount of a molecule that inhibits binding of an angiogenic chemokine to DARC. DARC is well known to those in the art. See background section for a brief description of DARC.

DARC inhibited in the methods of the present invention may be any DARC present on any cell, especially endothelial cells and epithelial cells. Examples of endothelial cells include, but are not limited to, vein endothelial cells, artery endothelial cells, and microvascular endothelial cells. Examples of epithelial cells include cells in lung alveoli and kidney collecting ducts.

DARC is capable of binding to chemokines, especially angiogenic chemokines. The term “angiogenic chemokine” as used herein refers to chemokines which stimulate or promote angiogenesis. The angiogenic chemokines can belong to any family of chemokines, such as C, CC, CXC, and CX₃C.

Examples of CC chemokines that can bind DARC and are angiogenic include, but are not limited to, monocyte-chemotactic protein-1 (MCP-1, also known as CCL2), MCP-3 (CCL7), RANTES (CCL5), and Eotaxin (CCL11).

The CXC chemokines can be subdivided into peptides that contain the sequence Glu-Leu-Arg (ELR) at their amino termini (ELR+), and those that do not (ELR−). Generally, angiogenic CXC chemokines are ELR+. Examples of angiogenic CXC ELR+chemokines that can bind DARC include Gro-α (CXCL1), Gro-P (CXCL2), ENA-78 (CXCL5), I-TAC (CXCL11), interleukin-8 (IL-8, also known as CXCL8), and homeostatic chemokines, such as TARC(CCL17).

Any molecule that inhibits binding of an angiogenic chemokine to DARC is useful in the methods of the present invention. Any mechanism of blocking binding may be employed. The molecule can, for example, block binding of the angiogenic chemokine to DARC by binding to DARC or to the angiogenic chemokine.

The molecule can be a small molecule or a biological molecule. Small molecules are typically organic compounds, including organometallic and organosilicon compounds, and the like, and generally have molecular weights of approximately 450 or less. Small molecules can further include molecules that would otherwise be considered biological molecules, except their molecular weights are not greater than 450. Thus, small molecules can include, monosaccharides, oligosaccharides, amino acids, oligopeptides, nucleotides, oligonucleotides, and their derivatives, having a molecular weight of 450 or less.

It is emphasized that a small molecule can have any molecular weight. They are merely called small molecules because they do not qualify as biological molecules, and typically have molecular weights less than 450.

Biological molecules are molecules which contain a polyamino acid, a polynucleotide, or a polysaccharide, and generally have a molecular weight of greater than 450. Polyamino acids include proteins, polypeptides, and peptides. Examples of polyamino acids useful in the methods of the present invention include antibodies that bind to DARC or to angiogenic chemokines, and that inhibit binding of angiogenic chemokines to DARC.

In this specification, an antibody is defined broadly as a protein that binds specifically to an epitope. Antibodies that bind specifically to an epitope may comprise an antibody hypervariable region. The protein that comprises an antibody hypervariable region may be a whole antibody or antibody fragment.

For example, the hypervariable region may comprise an entire antibody variable region. The antibody variable region may further comprise an antibody constant region. The antibody may be polyclonal or monoclonal.

Suitable variable and hypervariable regions of non-human antibodies may be derived from antibodies produced by any non-human mammal in which monoclonal antibodies are made. Suitable examples of mammals other than humans include, for example, rabbits, rats, mice, horses, goats, or primates.

Preferably, the antibodies are human antibodies. Human antibodies may be produced in a transgenic mouse. An example of such a mouse is the so-called XenoMouse™ (Abgenix, Freemont, Calif.) described by Green, LL., “Antibody Engineering Via Genetic Engineering of the Mouse: XenoMouse Stains are a Vehicle for the Facile Generation of Therapeutic Human Monoclonal Antibodies,” J. Immunol. Methods,” 10; 231(1-2):11-23(1999).

Antibody fragments have binding characteristics that are the same as, or are comparable to, those of the whole antibody. Suitable fragments of the antibody include any fragment that comprises a sufficient portion of the hypervariable (i.e. complementary determining) region to bind specifically, and with sufficient affinity to, for example, DARC or an angiogenic chemokine.

The preferred fragments are single chain antibodies. Single chain antibodies are polypeptides that comprise at least the variable region of the heavy chain of the antibody and the variable region of the light chain, with or without an interconnecting linker.

A non-human antibody may be chimerized. A chimerized antibody comprises the variable region of a non-human antibody and the constant region of a human antibody.

A non-human antibody is more preferably a humanized antibody. A humanized antibody comprises the hypervariable region (CDRs) of a non-human antibody. The variable region other than the hypervariable region, e.g. the framework variable region, and the constant region of a humanized antibody are those of a human antibody.

The antibodies may be members of any class of immunoglobins, such as: IgG, IgM, IgA, IgD or IgE, and the subclass(es) thereof.

The antibodies can be made by any method known to those in the art. Methods for making monoclonal antibodies include, for example, the immunological method described by Kohler and Milstein 1975. Nature 256:495-497 and by Campbell in “Monoclonal Antibody Technology, The Production and Characterization of Rodent and Human Hybridomas” in Burdon, et al., Eds, Laboratory Techniques in Biochemistry and Molecular Biology, Volume 13, Elsevier Science Publishers, Amsterdam (1985). The recombinant DNA method described by Huse, et al. 1989 Science 246:1275-1281 is also suitable.

Antibodies or antibody fragments can be isolated from antibody phage libraries generated using techniques, for example, described in McCafferty et al. 1990. Nature 348: 552-554, using the antigen of interest to select for a suitable antibody or antibody fragment. Clackson et al. 1991. Nature 352: 624-628 and Marks et al. 1991. J. Mol. Biol. 222: 581-597 describe the isolation of murine and human antibodies, respectively, using phage libraries. Subsequent publications describe the production of high affinity (nM range) human antibodies by chain shuffling (Mark et al. 1992. Bio/Technol. 10: 779-783), as well as combinatorial infection and in vivo recombination as a strategy for constructing very large phage libraries (Waterhouse et al. 1993. Nuc. Acids Res. 21: 2265-2266).

Methods for making chimeric and humanized antibodies are also known in the art. For example, methods for making chimeric antibodies include those described in U.S. patents by Boss (Celltech) and by Cabilly (Genentech). See U.S. Pat. Nos. 4,816,397 and 4,816,567, respectively. Methods for making humanized antibodies are described, for example, in Winter, U.S. Pat. No. 5,225,539.

The preferred method for the humanization of antibodies is called CDR-grafting. In CDR-grafting, the regions of a non-human mammalian antibodies, preferably a mouse antibody, that are directly involved in binding to antigen, the complementarity determining region or CDRs, are grafted into human variable regions to create “reshaped human” variable regions. These fully humanized variable regions are then joined to human constant regions to create complete “fully humanized” antibodies.

In order to create fully humanized antibodies that bind well to an antigen, it is advantageous to design the reshaped human variable regions carefully. The human variable regions into which the CDRs will be grafted should be carefully selected, and it is usually necessary to make a few amino acid changes at critical positions within the framework regions (FRs) of the human variable regions.

Methods for making single chain antibodies are also known in the art. Such methods include screening phage libraries transfected with immunoglobulin genes described in U.S. Pat. No. 5,565,332; U.S. Pat. No. 5,5837,242; U.S. Pat. No. 5,855,885; U.S. Pat. No. 5,885,793; and U.S. Pat. No. 5,969,108. Another method includes the use of a computer-based system for designing linker peptides for converting two separate polypeptide chains into a single chain antibody described in U.S. Pat. No. 4,946,778; U.S. Pat. No. 5,260,203; U.S. Pat. No. 5,455,030; and U.S. Pat. No. 5,518,889.

Other methods for producing antibodies described above are disclosed by Wels et al. in European patent application EP 502 812 and Int. J. Cancer 60:137-144 (1995); PCT Application WO 93/21319; European Patent Application 239 400, PCT Application WO 89/09622; European Patent Application 338 745; U.S. Pat. No. 5,658,570; U.S. Pat. No. 5,693,780; and European Patent Application EP 332 424.

Other examples of biological molecules that inhibit binding of angiogenic chemokines to DARC include mutated angiogenic chemokines, such as mutants of the angiogenic CXC or CC chemokines described above. The term “mutated angiogenic chemokine” as used in this specification refers to an analog of a natural (e.g., nonmutated) angiogenic chemokine. The analog is capable of binding to DARC, but has decreased ability to stimulate or promote angiogenesis.

The ability of the mutated angiogenic chemokine to stimulate or promote angiogenesis is considered to be decreased if the angiogenesis is inhibited by at least about 10%, preferably at least about 25%, more preferably at least about 50%, even more preferably at least about 75%, and most preferably at least about 90% compared to that of the nonmutated angiogenic chemokine.

The angiogenic chemokine can be mutated by any method known to those skilled in the art. For example, the mutated angiogenic chemokine may comprise a fragment of the natural angiogenic chemokine.

The fragment of an angiogenic chemokine is active (e.g., capable of binding to DARC, but has decreased ability to stimulate or promote angiogenesis). The fragment can be from the N-terminus, the C-terminus, or between the N- and C-terminus of an angiogenic chemokine. The fragment can, for example, contain at least about 10%, preferably about 25%, more preferably about 50%, even more preferably about 75%, and most preferably about 90% of the amino acids in the angiogenic chemokine. For example, if the angiogenic chemokine is 100 amino acids in length, the fragment can contain at least about ten, preferably about 25, more preferably about 50, even more preferably about 75, and most preferably about 90 amino acids.

Alternatively, one or more amino acids of the natural angiogenic chemokine may be replaced with other amino acids. An example of a mutated angiogenic chemokine capable of binding to DARC is melanoma growth stimulating activity (MGSA) E6A. MGSA contains an alanine at amino acid position 6 instead of glutamic acid. MGSA E6A is disclosed in, for example, Hesselgesser et al., JBC, 1995, 270:11472-11476.

Further examples of biological molecules that inhibit binding of angiogenic chemokines to DARC include nonimmunogenic malaria parasite ligands. Ligands from the malaria parasite are capable of binding to DARC. Such ligands are known to those in the art and include, for example, Duffy binding protein (DBP, 135 kd) of malaria parasite Plasmodium vivax ligand or its equivalent of Plasmodium falciparum ligand (e.g., erythrocyte binding antigen-175 (EBA-175) containing Duffy binding like (DBL) domains.

The term “nonimmunogenic” as used in this specification refers to the property of analogs of ligands from the malaria parasite that are capable of binding to DARC, but that lack immunogenicity. Ligands from the malaria parasite can be rendered nonimmunogenic by any method known to those skilled in the art. For example, the nonimmunogenic malaria parasite ligand may comprise a fragment of an immunogenic malaria parasite ligand. Alternatively, one or more amino acids of the malaria parasite ligand may be replaced with other amino acids.

The fragment of a malaria parasite ligand is active (e.g., capable of binding to DARC, but is nonimmunogenic). The fragment can be from the N-terminus, the C-terminus, or between the N- and C-terminus of a malaria parasite ligand. The fragment can, for example, contain at least about 10%, preferably about 25%, more preferably about 50%, even more preferably about 75%, and most preferably about 90% of the amino acids in the malaria parasite ligand. For example, if the malaria parasite ligand is 100 amino acids in length, the fragment can contain at least about ten, preferably about 25, more preferably about 50, even more preferably about 75, and most preferably about 90 amino acids.

Other examples of molecules that inhibit binding of angiogenic chemokines to DARC include peptidomimetics of any of the polyamino acids described above. As used herein, the term “peptidomimetic” means a molecule, especially a biological molecule, that recreates the stereospatial properties of the binding elements of a polyamino acid.

In addition, peptidomimetics typically enhance some property of the original polyamino acid. Such properties include, for example, increased stability, increased binding, increased activity, increased efficacy, enhanced delivery, increased half life, etc. Methods of making peptidomimetics based upon a known polyamino acid sequence are described, for example, in U.S. Pat. Nos. 5,631,280; 5,612,895; and 5,579,250.

The synthesis of peptidomimetics can involve the incorporation into a polyamino acid, a non-amino acid residue with non-amide linkages at a given position. For example, to obtain a peptidomimetic of a polyamino acid, a bond, backbone or an amino acid residue can be replaced with a suitable mimic. Some non-limiting examples of unnatural amino acid residues which may be suitable amino acid mimics include β-alanine, L-α-amino butyric acid, L-γ-amino butyric acid, L-α-amino isobutyric acid, L-α-amino caproic acid, 7-amino heptanoic acid, L-aspartic acid, N-ε-Boc-N-α-CBZ-L-lysine, N-ε-Boc-N-α-Fmoc-L-lysine, L-methionine sulfone, L-norleucine, L-norvaline, N-α-Boc-N-δCBZ-L-ornithine, N-δ-Boc-N-α-CBZ-L-ornithine, Boc-p-nitro-L-phenylalanine, Boc-hydroxyproline, and Boc-L-thioproline.

Inhibiting Angiogenesis

In another embodiment, the invention provides a method for inhibiting angiogenesis in a mammal in need thereof. As discussed in the background section, angiogenesis refers to the growth of new blood vessels. The inhibition of angiogenesis can occur in any type of cell, especially endothelial cells and epithelial cells, such as those described above.

The method for inhibiting angiogenesis comprises administering to the mammal an effective amount of a molecule that inhibits binding of an angiogenic chemokine to DARC. The molecule, angiogenic chemokine, and DARC include those disclosed above.

Mammals in need of inhibiting angiogenesis are those mammals suffering from a disease or condition where angiogenesis is not beneficial to the mammal. Any such disease or condition can be treated in accordance with the method of the present invention. Examples of appropriate diseases or conditions include, but are not limited to, benign or malignant tumor growth, metastasis, abnormal ocular neovascularization, arthritis, psoriasis, and several disorders of the female reproductive system including excessive bleeding due to enhanced vascular growth in conditions such as endometriosis, dysmenorhea, endrometrial and uterine cancer.

Promoting Tumor Necrosis

In a further embodiment, the invention provides a method for promoting tumor necrosis in a mammal in need thereof. Tumor necrosis refers to death of the cells of a tumor. Typically, necrosis of a tumor occurs from lack of blood supply. Promotion of tumor necrosis can occur in any type of tumor that requires a blood supply, such as those described above.

The method for promoting tumor necrosis comprises administering to the mammal an effective amount of a molecule that inhibits binding of an angiogenic chemokine to DARC. The molecule, angiogenic chemokine, and DARC include those disclosed above.

Mammals in need of promoting tumor necrosis are those mammals suffering from a tumor. Any type of tumor that requires a blood supply can be treated in accordance with the method of the present invention. Examples of such tumors include those discussed above.

Screening Drug Candidates

In another embodiment, the invention relates to a method for screening for drug candidates that inhibit angiogenesis. A drug candidate is a molecule that has the potential to be a useful medicament, pending further biological tests.

The first step in the method for screening for drug candidates is contacting a molecule with DARC. The molecule can be any molecule, including those described above.

As discussed above, DARC is well known to those skilled in the art. DARC useful in the screening method can be present on a cell. Examples of cells which contain DARC include endothelial cells and epithelial cells, such as those discussed above, and erythroid cells.

The cell can, for example, be fixed ex vivo. Methods for fixing cells are well known to those in the art. Typically, cells are fixed with a solution containing formalin or paraformaldhyde. Cell membrane preparations comprising DARC may also be used.

Alternatively, DARC can be prepared in vitro by methods that are well known in the art. One such method includes isolating or synthesizing DNA encoding DARC, and producing the recombinant protein by expressing the DNA, optionally in a recombinant vector, in a suitable host cell. Other methods for preparing DARC include isolating DARC from cells or synthesizing DARC. Suitable methods for preparing DARC are described below.

The molecule and DARC can be contacted with each other by any method known to those skilled in the art. Typically, either DARC or the molecule is immobilized on a solid support.

For example, DARC may be immobilized on a solid support, such as on a resin in a column. DARC can be contacted with a molecule by eluting the molecule through the column containing DARC immobilized on the resin.

Alternatively, the molecule may be immobilized on a solid surface, such as on a well of a microtitre plate. DARC can be contacted with the molecule by adding DARC into the well and incubating the plate. Many different molecules may be immobilized on a plate, thereby allowing the rapid screening of the molecules.

The next step in screening is to determine whether the molecule binds to DARC. Binding can be determined by any method known in the art.

For example, a label may be bound to the molecule or to DARC, depending on which is immobilized to the solid support. Usually, the component that is not immobilized is the component that is labeled. Thus, if the molecule is immobilized, DARC is labeled. If DARC is immobilized, the molecule is labeled.

After contacting the molecule and DARC, detection of binding of the molecule and DARC, for example by detecting the label, indicates that the molecule is a drug candidate that inhibits angiogenesis.

The method for screening for drug candidates optionally comprises the further step of determining whether the drug candidate inhibits angiogenesis. Any method known to those skilled in the art can be employed to determine whether the drug candidate inhibits angiogenesis.

For example, an in vitro culture of endothelial cells can be incubated with the drug candidate. The culture can then be assayed, by any method known to those in the art, to determine whether the drug candidate inhibited proliferation and migration of the endothelial cells and formation of capillary compared to a control culture without the drug candidate.

Alternatively, in vivo angiogenesis assays can be employed. Such assays are well known to those skilled in the art. For instance, a matrigel can be implanted into a mammal, such as a rat, followed by administration of a drug candidate or control compound. After incubation for a given period, the matrigel is removed and assayed to determine whether blood vessels are present in the matrigel in the treated animals and compared to the control. A smaller amount of blood vessels present or no vessels present in the matrigel of animals treated with the drug candidate compared to that of the control animals indicate that the drug candidate inhibits angiogenesis.

Angiogenesis is considered inhibited if the angiogenesis is inhibited by at least about 10%, preferably at least about 25%, more preferably at least about 50%, even more preferably at least about 75%, and even more preferably at least about 90%. Optimally, angiogenesis is inhibited 100%.

Administration of Molecule

The effective amount of a molecule administered in accordance with the methods of the invention is any amount effective for its purpose, e.g., inhibiting tumor growth, inhibiting angiogenesis or promoting tumor necrosis. Such effective amounts are those amounts which impart a beneficial effect (e.g., inhibit tumor growth, inhibit angiogenesis or promote tumor necrosis).

The actual amounts of a molecule will vary according to various factors that are well known in the art, such as the particular molecule utilized, the mode of application, particular subject to be treated, the size of the tumor, the degree of angiogenesis, etc. The appropriate amount of the molecule can readily be determined by those skilled in the art during preclinical and clinical trials.

The minimum amount of a molecule administered to a mammal is the lowest amount capable of achieving its purpose. The maximum amount administered to a mammal is the highest effective amount that does not cause undesirable side effects.

A molecule is considered to inhibit the binding of an angiogenic chemokine to DARC if the molecule causes a significant reduction in such binding. A molecule is considered to inhibit tumor growth or to inhibit angiogenesis if the molecule causes a significant reduction in tumor growth or angiogenesis. A reduction in binding, tumor growth or angiogenesis is considered significant, for example, if the binding, tumor growth or angiogenesis is at least about 10%, preferably at least about 25%, more preferably at least about 75%, and most preferably at least about 90% of the binding, tumor growth or angiogenesis is inhibited in the absence of the molecule.

In another embodiment, a molecule is considered to cause a significant promotion in tumor necrosis if the size of the tumor is reduced by at least about 10%, preferably at least about 25%, more preferably at least about 75%, and most preferably at least about 90%.

Any mammal can be treated in accordance with the methods of the present invention. Mammals include, for example, humans, baboons, and other primates, as well as pet animals such as dogs and cats, laboratory animals such as rats and mice, and farm animals such as horses, sheep and cows.

The molecule may be administered by any method known in the art. Some examples of suitable modes of administration include oral and systemic administration. Systemic administration can be enteral or parenteral. Liquid or solid (e.g., tablets, gelatin capsules) formulations can be employed.

Parenteral administration of the molecule include, for example intravenous, intramuscular, and subcutaneous injections. For instance, a molecule may be administered to a mammal by sustained release, as is known in the art. Sustained release administration is a method of drug delivery to achieve a certain level of the drug over a particular period of time.

Other routes of administration include oral, topical, intrabronchial, or intranasal administration. For oral administration, liquid or solid formulations may be used. Some examples of formulations suitable for oral administration include tablets, gelatin capsules, pills, troches, elixirs, suspensions, syrups, and wafers. Intrabronchial administration can include an inhaler spray. For intranasal administration, administration of a molecule can be accomplished by a nebulizer or liquid mist.

The molecule can be formulated in a suitable pharmaceutical carrier. In this specification, a pharmaceutical carrier is considered to be synonymous with a vehicle or an excipient as is understood by practitioners in the art. Examples of carriers include starch, milk, sugar, certain types of clay, gelatin, stearic acid or salts thereof, magnesium or calcium stearate, talc, vegetable fats or oils, gums and glycols.

The molecule can be formulated into a composition containing one or more of the following: a stabilizer, a surfactant, preferably a nonionic surfactant, and optionally a salt and/or a buffering agent.

The stabilizer may, for example, be an amino acid, such as for instance, glycine; or an oligosaccharide, such as for example, sucrose, tetralose, lactose or a dextran. Alternatively, the stabilizer may be a sugar alcohol, such as for instance, mannitol; or a combination thereof. Preferably the stabilizer or combination of stabilizers constitutes from about 0.1% to about 10% weight for weight of the molecule.

The surfactant is preferably a nonionic surfactant, such as a polysorbate. Some examples of suitable surfactants include Tween 20, Tween 80; a polyethylene glycol or a polyoxyethylene polyoxypropylene glycol, such as Pluronic F-68 at from about 0.001% (w/v) to about 10% (w/v).

The salt or buffering agent may be any salt or buffering agent, such as for example sodium chloride, or sodium/potassium phosphate, respectively. Preferably, the buffering agent maintains the pH of the molecule formulation in the range of about 5.5 to about 7.5. The salt and/or buffering agent is also useful to maintain the osmolality at a level suitable for administration to a mammal. Preferably the salt or buffering agent is present at a roughly isotonic concentration of about 150 mM to about 300 mM.

The molecule can be formulated into a composition which may additionally contain one or more conventional additives. Some examples of such additives include a solubilizer such as, for example, glycerol; an antioxidant such as for example, benzalkonium chloride (a mixture of quaternary ammonium compounds, known as “quart”), benzyl alcohol, chloretone or chlorobutanol; anaesthetic agent such as for example a morphine derivative; or an isotonic agent etc., such as described above. As a further precaution against oxidation or other spoilage, the composition may be stored under nitrogen gas in vials sealed with impermeable stoppers.

General Methods for Preparing DARC

Nucleic acids encoding DARC may be synthesized in vitro. The cDNA for DARC is disclosed in, for example, Chaudhuri, et al., PNAS 1993, 90:10793-10797. Suitable methods for synthesizing DNA are described by Caruthers et al. Science 1985, 230:281-285 and DNA Structure, Part A: Synthesis and Physical Analysis of DNA, Lilley, D. M. J. and Dahlberg, J. E. (Eds.), Methods Enzymol., 211, Academic Press, Inc. New York (1992).

DARC DNA may be replicated and expressed in a suitable host cell. Suitable host cells include prokaryotic host cells and eukaryotic host cells. A suitable prokaryotic host cell is E. coli. Suitable eukaryotic host cells include yeast cells, insect cells and mammalian cells.

The DARC may be isolated from, for example, cell membrane fractions by standard methods of protein isolation and purification. Some suitable methods include precipitation and liquid/chromatographic protocols such as, for instance, high performance liquid chromatography (HPLC), ion exchange, hydrophobic interaction chromatography, immunoprecipitation, lipid extraction, affinity chromatography and gel filtration, etc. See, for example, Guide to Protein Purification, Deutscher, M. P. (Ed.) Methods Enzymol., 182, Academic Press, Inc., New York (1990) and also Scopes, R. K. and Cantor, C. R. (Eds), Protein Purification (3d), springer-Verlag, New York (1994).

DARC may also be made synthetically, i.e. from individual amino acids, or semisynthetically, i.e. from oligopeptide units or a combination of oligopeptide units and individual amino acids. Suitable methods for synthesizing proteins are described by Stuart and Young in “Solid Phase Peptide Snthesis,” Second Edition, Pierce chemical Company (1984), Solid Phase Peptide Synthesis, Methods Enzymol., 298, Academic Press, Inc., New York (1997).

Many standard well known cloning and expression and isolation/purification techniques that reflect the state of the art in recombinant DNA and protein methods are described in detail in Sambrook & Russel, Molecular Cloning. A Laboratory Mamual, Third Edition, cold Spring Harbor Laboratory Press, Cold Spring Harbor, N.Y. (2001).

### EXAMPLES

### Example 1

### Materials and Methods

Mouse strains: Duffy knockout (DKO) mice construction has been previously described (Luo et al., Genome Research 1997, 7:932-941). Heterozygous knockout mice [Dfy(+/−)] were crossed to produce Dfy(+/+) and Dfy(−/−) homozygous siblings. Experiments were carried out with age and sex matched animals. C57BL/6J mice were purchased from Jackson Laboratory (Bar Harbor, Me.).

Development of Tumor: Lewis Lung Carcinoma (LLC) cells were grown at log phase, harvested and suspended in PBS at 1×10⁷ cells/ml. 100 μl of cell suspension was injected subcutaneously near the midline dorsum of each animal (n=5). Tumor growth and necrosis in each group of mice were monitored for fourteen days post injection. After sacrificing the mice, the tumors size was recorded by digital photography and weighed for total tumor growth.

Histochemistry: In order to evaluate necrosis and blood vessel formation, tumors were fixed in 10% phosphate buffered formalin. Tumors were embedded in paraffin according to standard histological procedures. Cross sections of tumor (5 μm thick) were stained with hematoxilin and eosin for histological study.

Antibody inhibition of Tumor: Three days after LLC cell injection, five groups of WT C57BL/6J mice (n=5) were injected intraperitoneally with either 1 ml of PBS, Preimmune sera, anti-Glycophorin (Gyp), anti-Kell or anti-Dfy serum (1:1 dilution). Tumors were allowed to grow for fourteen days. Mice were euthanized and tumors weighed. The average weights were plotted for comparison.

### Example 2

### Tumor Growth in Duffy Wild Type and Duffy Knock Out Mice

Solid tumors in both Dfy(+/+) (wild type, WT) and Dfy(−/−) (Duffy knockout, DKO) mice were developed by injecting Lewis lung carcinoma (LLC) cells. In the wild type and knockout mice, the tumor initially grew at the same rate. Interestingly, the tumors in the knockout mice started to exhibit signs of necrosis earlier than that of the wild type mice.

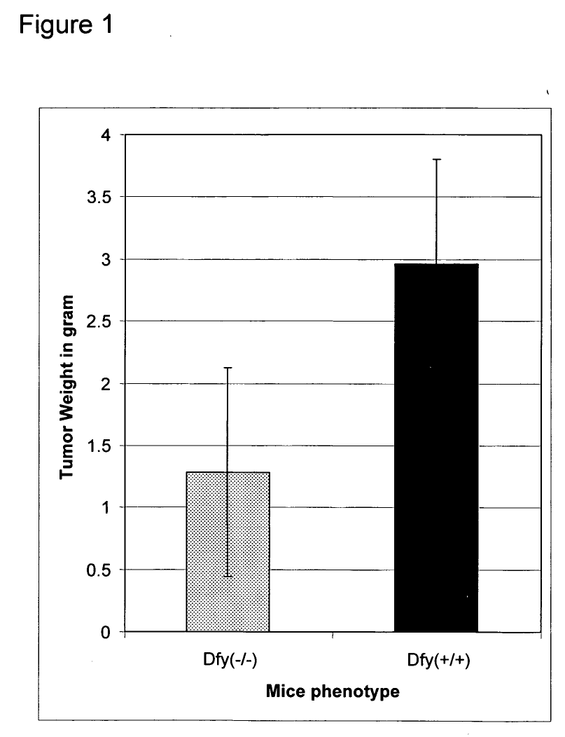

After fourteen days, the mice were sacrificed. The tumors were removed, weighed and compared. Results indicated that the solid tumors grew slower (by size and weight) in DARC knockout mice compared to that of wild type mice (FIG. 1).

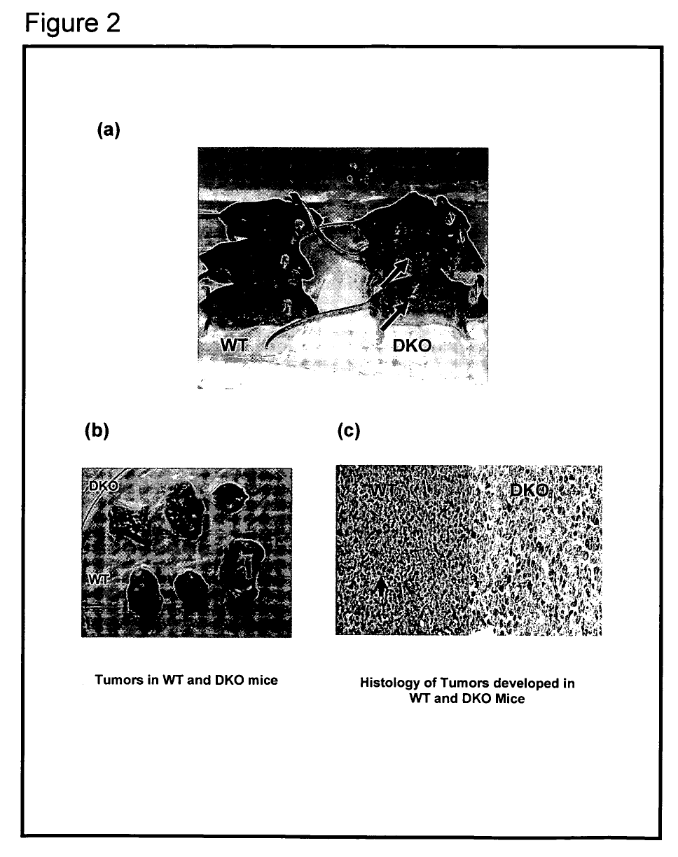

Tumors in knockout mice underwent severe necrosis (see arrow in FIG. 2a) while no necrosis was observed in tumors in wild type mice (FIG. 2a). There was more necrosis and vascular rapture in tumors in Duffy knockout mice compared to that in Dfy(+/+) mice (FIG. 2b). Moreover, immunohistological studies indicated that there was less microvascular capillary formation in the tumors of Duffy knockout mice compared to that in tumors of Dfy(+/+) mice (see arrow in FIG. 2c).

In order to determine whether antibody against Duffy protein had a similar effect in reducing tumor growth, we injected peritoneally anti-Duffy antibody made against the N-terminal domain of the protein. The tumor grew more slowly in antibody injected mice compared to PBS or preimmune serum injected mice group.

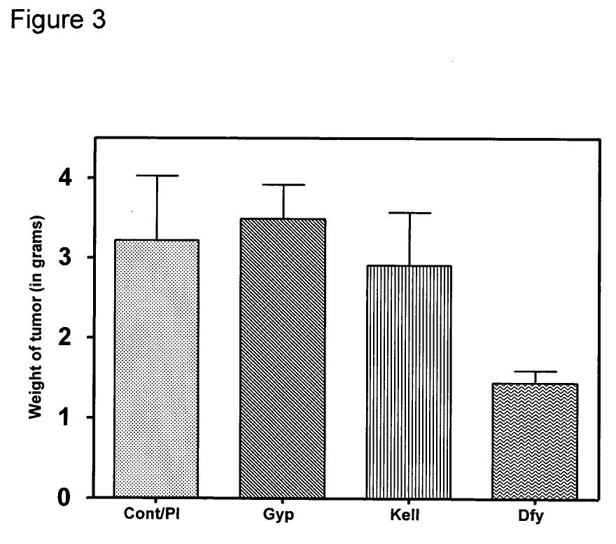

In order to determine that the antibody effect is not nonspecific, we injected mice group with antibodies against two other blood group antigens, such as glycophorin and Kell. Interestingly, none of the antibodies against the other blood group antigens had any effect on tumor growth (FIG. 3). Enhanced tumor necrosis was observed when wild type mice were injected with anti-Duffy antibody (data not shown).

The results demonstrate that DARC plays an important role in angiogenesis during tumor growth.

What is claimed is:

1. A method for screening for drug candidates that inhibit angiogenesis, the method comprising:
(i) contacting a molecule with a Duffy antigen receptor for chemokines; and
(ii) determining whether the molecule binds to the Duffy antigen receptor for chemokines;
wherein molecules that bind to the Duffy antigen receptor for chemokines are drug candidates that inhibit angiogenesis.

2. A method according to claim 1, further comprising determining whether the drug candidate inhibits angiogenesis.

3. A method according to claim 1, wherein the molecule is a small molecule.

4. A method according to claim 1, wherein the molecule is a biological molecule.

5. A method according to claim 4, wherein the biological molecule is a polyamino acid.

6. A method for inhibiting tumor growth in a mammal in need thereof, the method comprising administering to the mammal an effective amount of a molecule that inhibits binding of an angiogenic chemokine to Duffy antigen receptor for chemokines.

7. A method according to claim 6, wherein the molecule binds to a Duffy antigen receptor for chemokines.

8. A method according to claim 6, wherein the molecule is a biological molecule.

9. A method according to claim 8, wherein the biological molecule is a polyamino acid.

10. A method according to claim 9, wherein the polyamino acid is an antibody to Duffy antigen receptor for chemokines.

11. A method according to claim 6, wherein the mammal is a human.

12. A method according to claim 6, wherein the chemokine is a CXC chemokine.

13. A method according to claim 12, wherein the CXC chemokine is a CXC/ELR+chemokine.

14. A method according to claim 6, wherein the chemokine is a CC chemokine.

15. A method for inhibiting angiogenesis in a mammal in need thereof, the method comprising administering to the mammal an effective amount of a molecule that inhibits binding of an angiogenic chemokine to Duffy antigen receptor for chemokines.

16. A method for promoting tumor necrosis in a mammal in need thereof, the method comprising administering to the mammal an effective amount of a molecule that inhibits binding of an angiogenic chemokine to Duffy antigen receptor for chemokines.
